(19) United States
(12) Patent Application Publication
(10) Pub. No.: US 2025/0090069 A1
(43) Pub. Date: Mar. 20, 2025

(54) ELECTROANATOMICAL MAPPING DILATOR ELECTRICAL CONNECTION TO SHEATH

(71) Applicant: Boston Scientific Scimed, Inc., Maple Grove, MN (US)

(72) Inventor: Jeffery D. Arnett, Gilbert, AZ (US)

(21) Appl. No.: 18/883,937

(22) Filed: Sep. 12, 2024

Related U.S. Application Data
(60) Provisional application No. 63/582,657, filed on Sep. 14, 2023.

(51) Int. Cl. A61B 5/287; A61B 5/339; A61B 5/367; A61M 29/00

(52) U.S. Cl. CPC A61B 5/287; A61B 5/339; A61B 5/367; A61M 29/00; A61M 2205/0233; A61M 2205/0238

(57) ABSTRACT

A medical system includes a sheath including an elongate body having a proximal portion and a distal portion. One or more sheath electrodes is located on the distal portion. A handle is attached to the proximal portion, the handle includes a first connector capable of electrically coupling to a control system and a second connector. The system includes a dilator having an elongate body including a proximal portion and a distal portion having one or more dilator electrodes. The dilator includes a hub having a dilator connector electrically coupled to the one or more dilator electrodes. The second connector includes a retaining track having at least one electrical contact. The second connector is configured to mechanically and electrically couple with the dilator connector.

### CROSS REFERENCE TO RELATED APPLICATIONS

The present application claims priority to U.S. Provisional Patent Application No. 63/582,657, filed Sep. 14, 2023, the entire disclosure of which is incorporated herein by reference.

### TECHNICAL FIELD

The present invention relates generally to methods and devices usable within the body of a patient. More specifically, the present invention is concerned with an apparatus pertaining to an electroanatomical mapping enabled dilator and an electroanatomical mapping sheath having an integrated electrical coupling.

### BACKGROUND

Electroanatomical mapping (EAM) is an increasingly prevalent technology useful during in vivo procedures. It enables physicians to identify anatomical regions of the heart and patterns of electrical activation. This is especially useful when treating arrhythmias. Devices that are compatible with EAM systems allow operators to localize them and more easily target specific regions for treatment, allowing for better workflow, better treatment efficacy, and shorter procedure time.

Typically, each EAM device requires a cable to connect to the EAM system. This can result in cumbersome manipulation of the device and a challenging workspace to manage with several cables for the procedure's devices.

### SUMMARY

Example 1 is a medical system including a dilator having an elongate body having a proximal portion and a distal portion having one or more dilator electrodes, the dilator having a hub including a dilator connector electrically coupled to the one or more dilator electrodes. The system includes a sheath including an elongate body having a proximal portion and a distal portion, one or more sheath electrodes located on the distal portion and a handle attached to the proximal portion, the handle having a first connector capable of electrically coupling to a control system and a second connector. The second connector includes a retaining track having at least one electrical contact, the second connector being configured to mechanically and electrically couple with the dilator connector.

Example 2 is the system of Example 1, wherein the sheath elongate body includes a lumen extending from the proximal portion to the distal portion and is configured to receive the dilator.

Example 3 is the system of Example 2, wherein the second connector is coaxial with the lumen.

Example 4 is the system of any of Examples 1-3, wherein the resilient retaining track includes an oval shape body having a width greater than a height.

Example 5 is the system of Example 4, wherein the resilient retaining track includes a first line of symmetry and a second line of symmetry, and the at least one electrical contact is intersected by the first line of symmetry.

Example 6 is the system of Example 5, wherein the at least one electrical contact includes a conductive coating.

Example 7 is the system of any of Examples 1-6, wherein the second connector includes a hemostatic valve, a cap, and a cover.

Example 8 is the system of Example 7, wherein the cap includes a retaining track guide.

Example 9 is the system of any of Examples 1-8, wherein electrical connection is made between the one or more dilator electrodes and the control system when the dilator connector is received by the second connector.

Example 10 is the system of any of Examples 1-9, wherein the dilator distal portion is tapered.

Example 11 is the system of any of Examples 1-10, wherein the dilator connector includes a cylindrical body having an outer surface, and one or more electrical contacts positioned on the outer surface.

Example 12 is the system of any of Examples 1-11, wherein the dilator connector is configured to mechanically couple with the second connector in order to maintain the dilator in a desired rotational orientation relative to the sheath.

Example 13 is the system of any of Examples 1-12, wherein the dilator connector includes a distal protrusion.

Example 14 is the system of any of Examples 1-13, further comprising a display for displaying one or more anatomical images, parameters, and positioning information.

Example 15 is the system of any of Examples 1-14, wherein the one or more sheath electrodes includes four sheath electrodes and the one or more dilator electrodes includes two electrodes.

Example 16 is medical system including a sheath having an elongate body having a proximal portion, a distal portion, and a lumen therebetween. One or more sheath electrodes is located on the distal portion. A handle is attached to the proximal portion. The handle includes a first connector capable of electrically coupling to a control system and a second connector coaxial with the lumen. The system includes a dilator. The dilator includes an elongate body having a proximal portion and a distal portion having one or more dilator electrode. The dilator has a dilator connector electrically coupled to the one or more dilator electrodes. The second connector includes a retaining track having at least one electrical contact. The second connector is configured to mechanically and electrically couple with the dilator connector.

Example 17 is the system of Example 16, wherein the sheath lumen is configured to receive the dilator.

Example 18 is the system of Example 17, wherein the dilator includes a dilator hub, and the dilator connector is integral with the dilator hub.

Example 19 is the system of Example 16, wherein the resilient retaining track includes an oval shape body having a width greater than a height.

Example 20 is the system of Example 19, wherein the resilient retaining track includes a first line of symmetry and a second line of symmetry, and the at least one electrical contact is intersected by the first line of symmetry.

Example 21 is the system of Example 20, wherein the at least one electrical contact includes a conductive coating.

Example 22 is the system of Example 16, wherein the second connector includes a hemostatic valve, a cap, and a cover.

Example 23 is the system of Example 22, wherein the cap includes a retaining track guide.

Example 24 is the system of Example 16, wherein electrical connection is made between the one or more dilator electrodes and the control system when the dilator connector is received by the second connector.

Example 25 is the system of Example 16, wherein the dilator distal portion is tapered.

Example 26 is the system of Example 16, wherein the dilator connector includes a cylindrical body having an outer surface, and one or more electrical contacts positioned on the outer surface.

Example 27 is the system of Example 16, wherein the dilator connector is configured to self-align with the second connector, and wherein the dilator connector is configured to mechanically couple with the second connector in order to maintain the dilator in a desired rotational orientation relative to the sheath.

Example 28 is the system of Example 16, wherein the dilator connector includes a distal protrusion.

Example 29 is the system of Example 16, further comprising a display for displaying one or more anatomical images, parameters, and positioning information.

Example 30 is the system of Example 16, wherein the one or more sheath electrodes includes four sheath electrodes and the one or more dilator electrodes includes two electrodes.

Example 31 is a medical system including a sheath including an elongate body having a proximal portion, a distal portion, and a lumen therebetween. One or more sheath electrodes is located on the distal portion. A handle is attached to the proximal portion. The handle has a first connector capable of electrically coupling to a control system and a second connector coaxial with the lumen. The system includes a dilator. The dilator has an elongate body including a proximal portion and a distal portion having one or more dilator electrodes. The dilator has a dilator connector electrically coupled to the one or more dilator electrodes. The second connector includes a resilient retaining track being configured to mechanically and electrically couple with the dilator connector. Electrical connection is made between the one or more dilator electrodes and the control system when the dilator connector is received by the second connector.

Example 32 is the system of Example 31, wherein the resilient retaining track includes an oval shape body having a width greater than a height.

Example 33 is the system of Example 32, wherein the resilient retaining track includes at least one electrical contact.

Example 34 is the system of Example 33, wherein the at least one electrical contact includes a conductive coating.

Example 35 is a medical system including a sheath including an elongate body having a proximal portion, a distal portion, and a lumen therebetween. One or more sheath electrodes is located on the distal portion. A handle is attached to the proximal portion, the handle includes a first connector capable of electrically coupling to a control system and a second connector coaxial with the lumen. The system includes a dilator including an elongate body having a proximal portion and a distal portion. One or more dilator electrodes located on the distal portion. A dilator connector is electrically coupled to the one or more dilator electrodes. The system includes a display for displaying one or more anatomical images, parameters, and positioning information. The second connector includes a resilient retaining track having at least one conductive snap. The second connector is configured to receive the dilator elongate body and to electrically couple with the dilator connector.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 illustrates a sheath in accordance with an embodiment of the present disclosure.

FIG. 3 illustrates a dilator in accordance with an embodiment of the present disclosure.

FIG. 4 illustrates the dilator of FIG. 3 inserted into the sheath of FIG. 2 in accordance with the present disclosure.

FIG. 5 illustrates an exploded view of a proximal sheath connector in accordance with an embodiment the present disclosure.

FIG. 6 illustrates a plan view of a retaining track, in accordance with an embodiment of the present disclosure.

FIG. 7 illustrates a plan view of a proximal sheath connector showing electrical contacts in accordance with an embodiment of the present disclosure.

FIG. 8 illustrates a dilator connector in accordance with the present disclosure.

FIG. 9 illustrates a cross-sectional view of a proximal sheath connector and a dilator connector prior to connection, in accordance with the present disclosure.

FIG. 10 illustrates a cross-sectional view of a proximal sheath connector and a dilator connector after connection, in accordance with the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

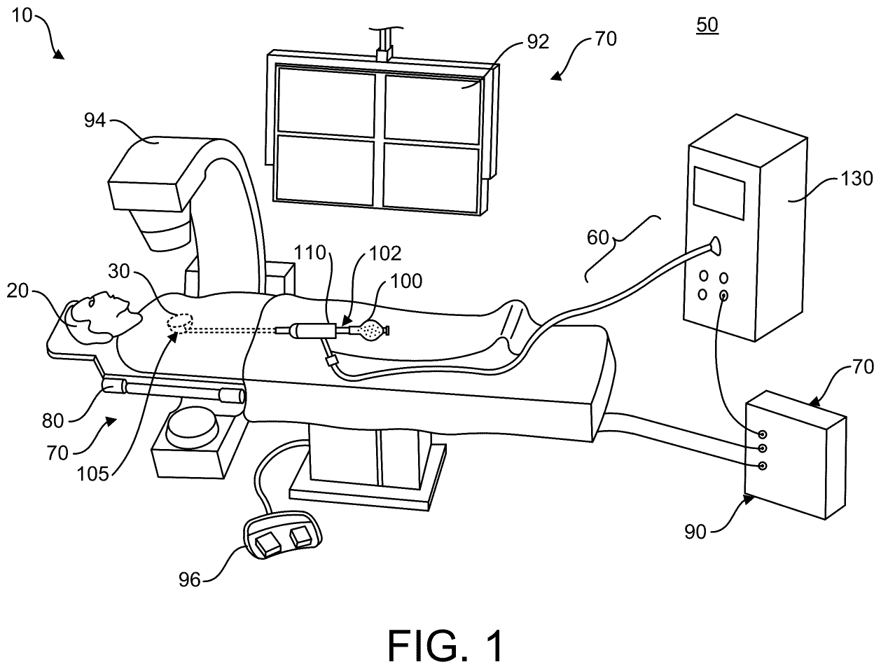

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an access system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The access system 60 includes a dilator 100 having a proximal portion 102 and a distal portion 105, an introducer sheath 110, and a console 130. In some aspects, the distal portion 105 includes a tapered region. The introducer sheath 110 includes a plurality of electrodes on a distal portion thereof. The introducer sheath 110 is connected to the EAM system using one or more cables, umbilicals, and the like, that operate to functionally connect the electrodes to the EAM system 70. The dilator 100 additionally includes a plurality of electrodes along the distal portion 105 for connecting to the EAM system 70. The dilator 100 does not directly connect to the EAM system 70 via a cable or umbilical. Rather, the dilator 100 connects to the EAM system 70 when inserted into the introducer sheath 110 as discussed in detail below.

In embodiments, the introducer sheath 110 is operable to provide a delivery conduit through which the dilator 100, in particular all or part of the distal portion 105 thereof, can be deployed to the specific target sites within the patient's heart 30. The dilator 100 is configured with a lumen such that a guiding device, for example a guidewire, or perforation device, for example an RF perforation device, can be inserted therein. In some aspects, a perforation device can be used to perform a transseptal crossing procedure within the heart 30.

The console 130 is configured to control functional aspects of the access system 60. In embodiments, the console 130 includes one or more controllers, microprocessors, and/or computers that execute code out of memory to control and/or perform the functional aspects of the access system 60. In embodiments, the memory can be part of the one or more controllers, microprocessors, and/or computers, and/or part of memory capacity accessible through a network, such as the world wide web. In embodiments, the console 130 can include pulse generator hardware, software and/or firmware configure to generate electrical pulses in predefined waveforms, which can be transmitted to electrodes positioned on a the dilator 100, guiding device, or perforation device to generate electric fields sufficient to achieve the desired clinical effect, for example ablation of target tissue through irreversible electroporation. In embodiments, the console 130 can deliver the pulsed waveforms in a monopolar or bipolar mode of operation, as will be described in further detail herein.

The EAM system 70 is operable to track the location of the various functional components of the access system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the dilator 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors. In some aspects, the EAM system 70 may include a display for displaying one or more anatomical images, parameters, and positioning information.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the dilator 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

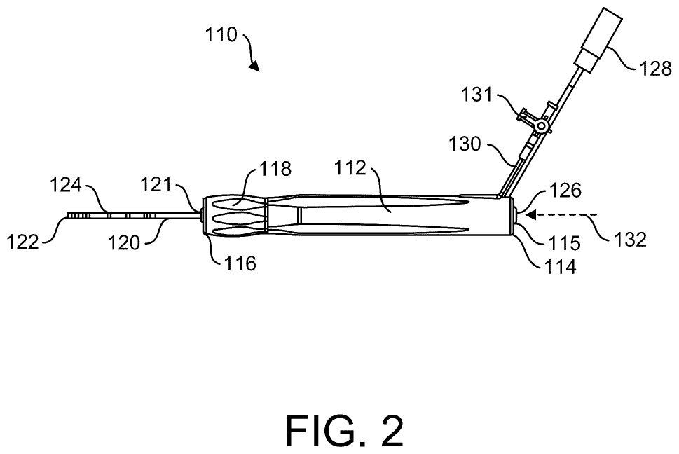

FIG. 2 illustrates a sheath 110 in accordance with an embodiment of the present disclosure. The sheath 110 includes a handle 112 configured to be gripped by a user to manipulate the sheath 110. The handle 112 includes a proximal end 114 and a distal end 116. A proximal sheath connector 115 is located at the proximal end 114 of the handle 112 and is configured to mate with a dilator connecter as discussed below.

A control knob 118 is located on the handle 112 and is rotatable to control the shape of an elongate hollow body 120 that extends from the handle 112. While the control knob 118 is illustrated near the distal end 116, it is understood that the control knob 118 may be located along any portion of the handle 112. The control knob 118 is configured to manipulate one or more steering wire or rod to change the shape of the elongate hollow body 120 and is configured to move relative to a stationary portion of the handle 112. The control knob 118 is configured to deflect the elongate hollow body 120 in a first direction when the knob 118 is rotated clockwise and to deflect the elongate hollow body 120 in a second direction when the knob 118 is rotated counterclockwise.

The elongate hollow body 120 includes a proximal end 121 and a distal end 122. The proximal end 121 is removably connected to the distal end 116 of the handle 112 and the distal end 122 is free to move. The elongate hollow body 120 includes one or more electrodes 124 positioned along a length thereof. In one aspect, the one or more electrodes 124 includes at least four electrodes. The one or more electrodes 124 can be evenly spaced or positioned at uneven intervals. The one or more electrodes 124 can be configured as surface electrodes capable of contacting tissue or fluid within a patient. The one or more electrodes 124 may be configured as part of an EAM system 70, to detect a parameter when in contact with the tissue or fluid, or to deliver energy to the tissue, such as RF energy in order to ablate the tissue.

A lumen 126 extends from the handle proximal end 114 though the knob 118 and through the elongate hollow body 120 to the distal end 122. The lumen 126 is configured to receive a dilator or other elongate medical device in a direction as indicated by arrow 132. The proximal sheath connector 115 is coaxial with the lumen 126 and is configured to receive a medical device therein, for example the elongate hollow body 142 of dilator 100.

The handle 112 includes a connector 128 that is configured to couple with a control system, for example an EAM system 70 or RF energy generator. The connector 128 is configured to releasably engage with the system. The handle 112 also includes a conduit 130 which includes at least one fitting 131, such as a Luer connector. The conduit 130 is configured to allow for introduction of fluids into the lumen 126 or an additional lumen within the sheath 110. The at least one fitting 131 is configured to removably connect to a syringe or other container for delivering a fluid through the conduit 130.

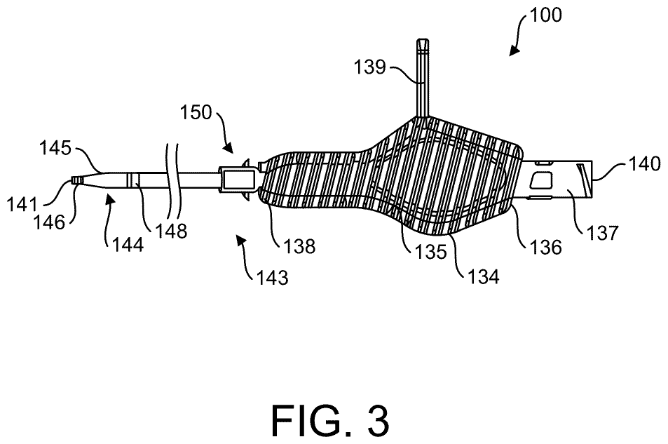

FIG. 3 illustrates a dilator 100 in accordance with an embodiment of the present disclosure. The dilator 100 is configured to be inserted into and couple with the sheath 110. The dilator 100 includes a hub 134. The hub 134 includes a knurled surface 135 configured to aid a user in gripping the hub 134. The hub 134 includes a proximal end 136 and an opposite distal end 138. A connector 137 is located at the proximal end 136. The connector 137 may include a Luer connector configured to removably connect to a syringe or other container. The connector 137 also allows for introduction of a medical device, such as a guidewire or RF perforation device, into a lumen 140 that extends from the connector 137 to a distal tip 141 of the dilator 100. The hub 134 includes an extension 139 that aids in manipulation of the dilator 100 and acts as a reference for positioning the dilator 100.

The dilator 100 includes an elongate hollow body 142 having a proximal portion 143 and a distal portion 144. The distal portion 144 includes a tapered section 145 including one or more electrodes 146, 148 positioned thereon. In the embodiment illustrated in FIG. 3, two electrodes 146, 148 are included on the tapered section 145.

The elongate hollow body 142 ends in distal tip 141. The one or more electrodes 146, 148 can be evenly spaced or positioned at uneven intervals. The one or more electrodes 146, 148 can be configured as surface electrodes capable of contacting tissue or fluid within a patient. The one or more electrodes 146, 148 may be configured to detect a parameter when in contact with the tissue or fluid or to deliver energy to the tissue, such as RF energy in order to ablate the tissue. The one or more electrodes 146, 148 are configured to electrically connect to a system, for example EAM system 70.

The one or more electrodes 146, 148 may be electrically coupled with a system, for example EAM system 70 when the dilator 100 is inserted into introducer sheath 110. In one embodiment, the one or more electrodes 146, 148 are electrically coupled with EAM system 70 when a dilator connector 150 is coupled to the proximal sheath connector 115. The dilator connector 150 is located at the proximal portion 143 of the elongate hollow body 142 just distal to the hub 134. In one embodiment, the dilator connector 150 is integral with the hub 134 and forms a portion thereof. In another embodiment, the dilator connector 150 is separate from the hub 134.

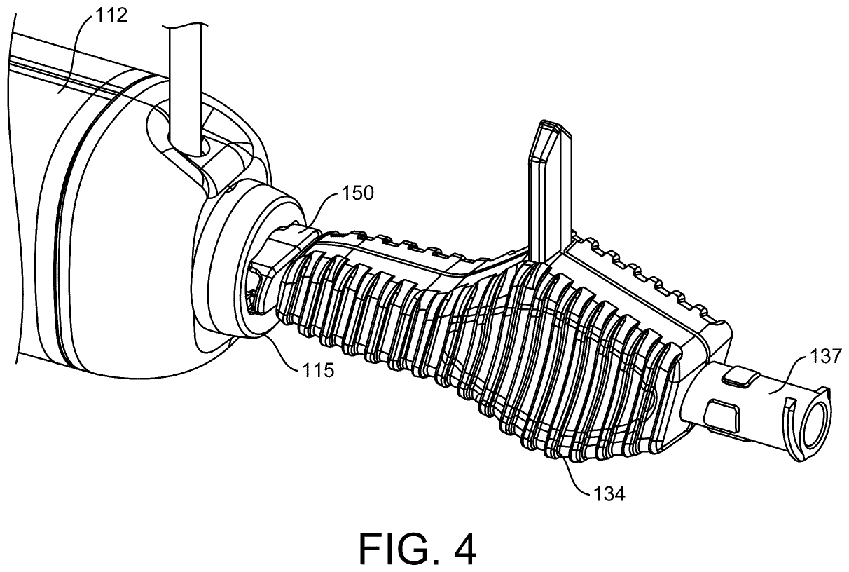

FIG. 4 illustrates the dilator 100 of FIG. 3 inserted into the introducer sheath 110 of FIG. 2 in accordance with the present disclosure. In FIG. 4, the dilator connector 150 is shown inserted into the proximal sheath connector 115. In this configuration, each of the one or more electrodes 124 of the introducer sheath and the one or more electrodes 146, 148 of the dilator 100 are electrically coupled to a control system, for example EAM system 70 or an energy generator. The dilator 100 has a length such that when dilator connector 150 is received in the proximal sheath connector 115, at least the distal portion 144 including the tapered section 145 and the one or more electrodes 146, 148 extend from the distal end 122 of the introducer sheath 110.

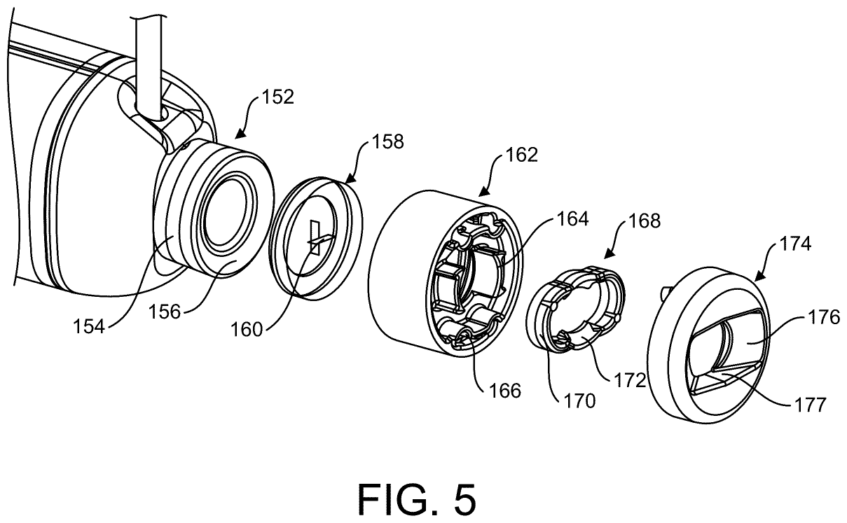

FIG. 5 illustrates an exploded view of a proximal sheath connector 115 in accordance with an embodiment the present disclosure. The proximal sheath connector 115 is configured to releasably connect with the dilator connector 150. Connecting the dilator 100 with the introducer sheath 110 allows a user to manipulate both the dilator 100 and sheath 110 together, as well as electrically connect the one or more electrodes 146, 148 to a system. The proximal sheath connector 115 includes a hub 152, hemostatic valve 158, cap 162, retaining track 168, and cover 174.

The hub 152 includes an outer surface 154 that is configured to connect with an inner surface of cap 162. The outer surface 154 may be include an element configured to interact with an element on the inner surface of cap 162. The elements may be configured to form a snap-fit, friction fit, or threaded connection. The hub 152 includes a face that includes a recess or channel 156 that is configured to mate with or hold the hemostatic valve 158. During assembly, the hemostatic valve 158 is placed within the recess or channel 156 and is held in place by cap 162. The hemostatic valve 158 includes an opening 160 that allows for the passage of a medical device into the sheath 110, but prevents fluids from leaving the sheath 110.

The cap 162 includes a proximal facing surface that includes a retaining track guide 164 and a cover connector 166. The retaining track guide 164 is configured to hold the retraining track 168 in proper location when assembled. The retaining track guide 164 allows the retaining track 168 to move freely within the cap 162 between a first configuration and a second configuration. The cover connector 166 is configured to connect with a corresponding connector of the cover 174. The connector 166 may be configured to form a snap-fit, friction fit, or threaded connection with the cover 174.

The retaining track 168 is a flexible or resilient component that is configured to retain a portion of the dilator 100 when inserted therein. The retaining track 168 includes a resilient oval shape body 170 and includes two snaps 172 which grip the dilator 100. The snaps provide the function of mechanically and electrically coupling with dilator 100 via dilator connector 150. The resilient oval shape body 170 is formed of a polymeric or metallic material to provide the proper force to retain the dilator 100 when inserted therein. The snaps 172 are provided with a conductive coating, film, or surface, or are made entirely of a conductive material to create an electrical contact. Each electrical contact of the snaps 172 are electrically connected to the connector 128 via one or more leads such that snaps 172 can be electrically connected to the EAM system 70 or other energy delivery system.

The cover 174 includes an opening to allow the dilator 100 or other medical device to be inserted into the sheath 110. The opening includes angled surfaces 176 and planar surfaces 177. The angled surfaces 176 and planar surfaces 177 are configured such that they provide self-alignment with dilator connecter 150 when the dilator 100 is inserted into the sheath 110. Additionally, the angled surfaces and the planar surfaces 177 are configured to maintain the dilator 100 in a desired rotational orientation relative to the sheath 110 when the dilator connector 150 is coupled with the proximal sheath connector 115.

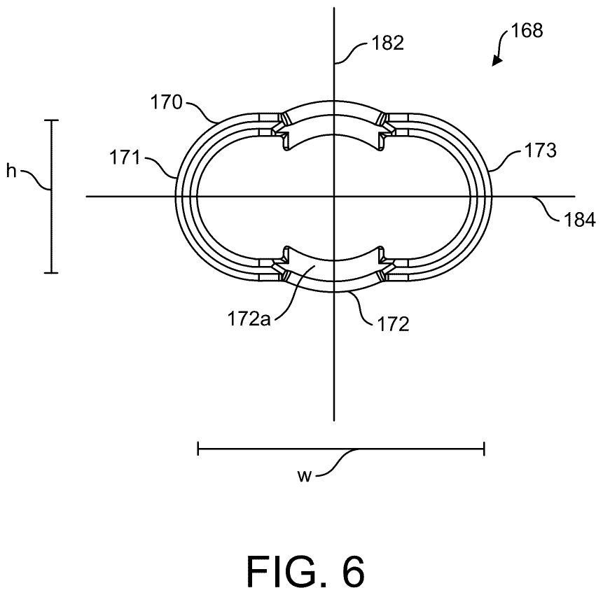

FIG. 6 illustrates a plan view of the retaining track 168. The resilient retaining track includes an oval shape body 170 having a width w greater than a height h. The oval shape body 170 includes a first curve 171 and a second curve 173 separated by snaps 172. Snaps 172 include an arcuate ramped surface 172a that extends towards a center of the oval shape body 170. The resilient retaining track 168 includes a first line of symmetry 182 and a second line of symmetry 184. The snaps 172 are arranged along the oval shape body 170 to be intersected by the first line of symmetry 182. In one embodiment, the snaps 172 can be formed of an electrically conductive material to form an electrical contact. In another embodiment, the snaps 172 may be formed of an electrically insulative material, but can include an electrically conductive coating or film to form an electrical contact. In one aspect, the arcuate ramped surface 172a may include a conductive coating or film.

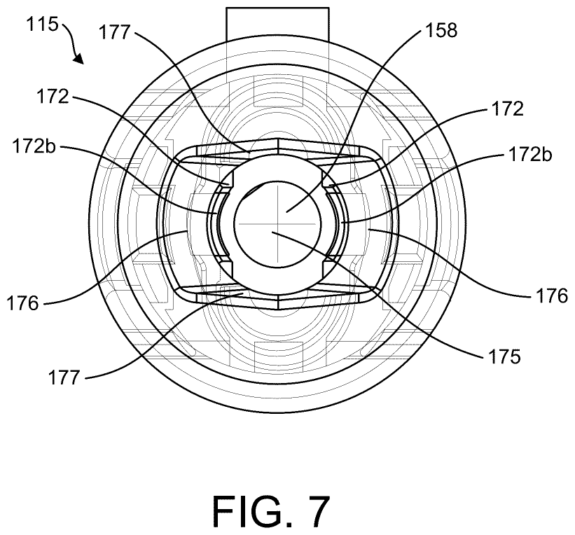

FIG. 7 illustrates a plan view of the proximal sheath connector 115 in accordance with an embodiment of the present disclosure. The retaining track 168 is shown in a first configuration, where no dilator is inserted into the connector 115. In this configuration, the snaps 172, along with the conductive material forming electrical contacts 172b, can be seen extending into the lumen 175. The electrical contact 172b may form only a portion of the snap 172 or may form the entire snap 172.

FIG. 7 show the angled surfaces 176 and planar surfaces 177 being arranged such that a distal tip 141 of the dilator 100 is directed toward the lumen 175. Hemostatic valve 185 can be seen positioned within the lumen.

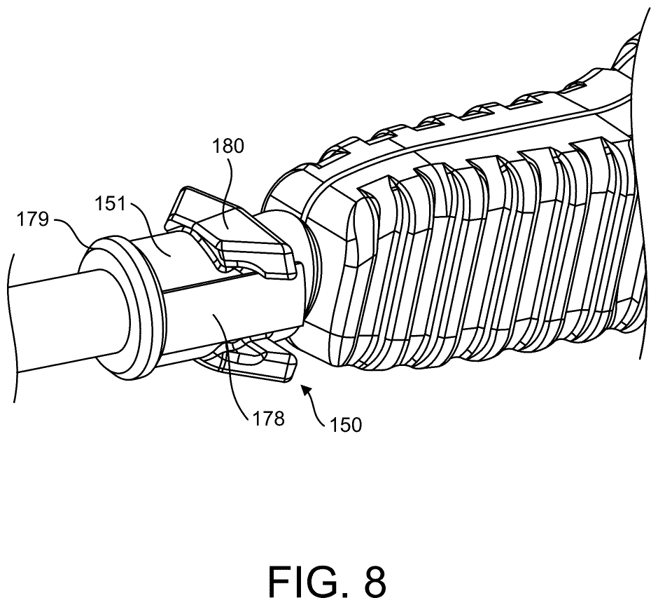

FIG. 8 illustrates a dilator connector 150 in accordance with the present disclosure. The dilator connector 150 includes a cylindrical body 151 having an outer surface. The cylindrical body 150 includes a distal protrusion 179 that forms a raised ring. The cylindrical body includes two ramped surfaces 180 that are separated by electrical contacts 178 on the outer surface of each side of the cylindrical body 151. The two ramped surfaces 180 interact with the angled surfaces 176 and planar surfaces 177 to provide self-alignment with dilator connecter 150 when the dilator 100 is inserted into the sheath 110.

In one embodiment, the electrical contacts 178 can be formed by a film or coating of electrically conductive material placed on a surface of the dilator connector 150. In another embodiment, a separate electrically conductive material can be formed, such as by stamping, and attached to a surface of the dilator connector 150. In another embodiment, the dilator connector 150 can be formed of a conductive material and covered in insulation, except for the portions forming electrical contacts 178. The electrical contacts 178 are electrically connected to the one or more electrodes 146, 148 by a lead or wire that extends along the elongate hollow body 142. As such, when the dilator connector 150 is inserted into the proximal sheath connector 115, the retaining track 168 assumes a second expanded configuration as the distal protrusion 179 is inserted therein. The retaining track 168 returns towards the first configuration once the distal protrusion 179 is advanced beyond the retaining track 168, and the snaps 172 apply compression force to the electrical contracts 178. Thus, electrically connecting the one or more electrodes 146, 148 to the EAM system 70 or energy delivery system.

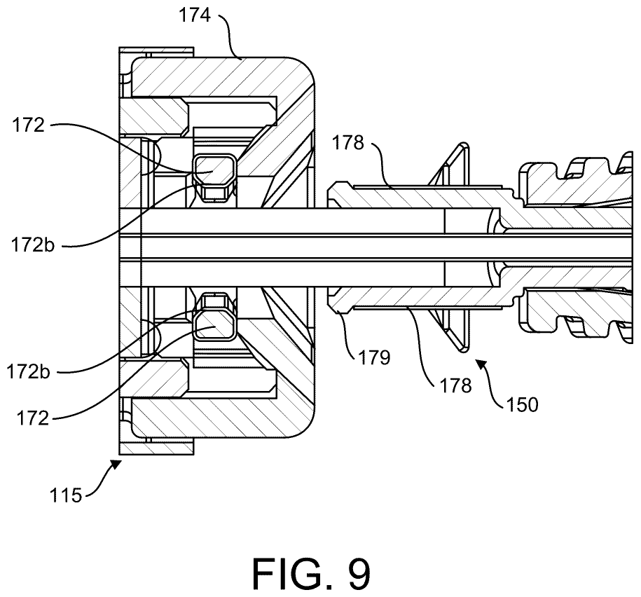

FIG. 9 illustrates a cross-sectional view of a proximal sheath connector 115 and a dilator connector 150 prior to connection, in accordance with the present disclosure. As can be seen in FIG. 9, the distal protrusion 179 of the dilator connector 150 is outside of the proximal sheath connector 115. As such, the dilator connector 150 electrical contacts 178 are not in contact with the sheath connector 115 electrical contacts 172b. Therefore, no electrical connection exists between the sheath connector 115 and the dilator connector 150.

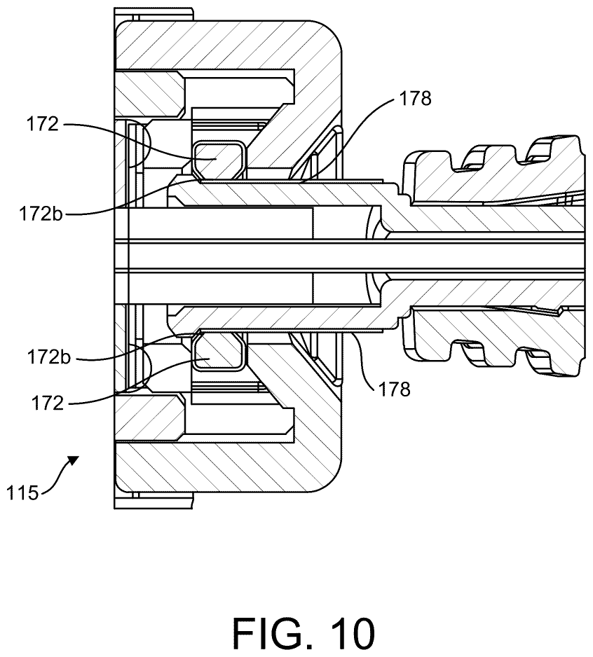

FIG. 10 illustrates a cross-sectional view of a proximal sheath connector 115 and a dilator connector 150 after mechanical and electrical connection, in accordance with the present disclosure. In FIG. 10, the dilator connector 150 has been inserted into the proximal sheath connector 115. The snaps 172 are in an engaged configuration and the electrical contacts 172b of the sheath connector 115 are in electrical communication with the electrical contacts 178 of the dilator connector 150. In this configuration, the one or more electrodes 146, 148 of the dilator 100 are electrically coupled with EAM system 70.

In some aspects, the sheath 110 and dilator 100 can be packaged as a kit and be ready for use right out of the package. Alternatively, the kit may include a plurality of sheaths or dilators each having different pre-shaped portions for use in various procedures.

In some aspects, the sheath 110 or dilator 100 may include one or more markers along a portion thereof for identifying a position or location during use using an imaging modality.

In some aspects, the sheath 110 or dilator 100 may include a plurality of cuts machined into the wall, for example by laser cutting. The shape and positioning of the cuts can allow for a transition in flexibility from a proximal portion to distal portion. The cuts may include a broken spiral configuration or may be positioned substantially orthogonal to a longitudinal axis of the sheath 110 or dilator 100. In some aspects, there may be a single cut that winds around an axis with a wider spacing between loops at the proximal portion and a larger spacing at the distal portion. The spacing and size of the cuts can be varied to achieve different flexibilities along the length of the sheath 110 or dilator 100.

In some aspects, the sheath 110 or dilator 100 may be formed of a shape memory material, such as a shape memory polymer or a shape memory metal. This would allow the sheath 110 or dilator 100 to have a first shape at a first temperature, and a second shape at a second temperature. Shape transition may be initiated by inserting a heated solution into the sheath 110 or dilator 100 or using electricity to heat a portion of the sheath 110 or dilator 100.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical system, comprising:
a sheath including an elongate body having a proximal portion, a distal portion, and a lumen therebetween;
one or more sheath electrodes located on the distal portion;
a handle attached to the proximal portion, the handle having a first connector capable of electrically coupling to a control system and a second connector coaxial with the lumen; and
a dilator including an elongate body having a proximal portion and a distal portion having one or more dilator electrodes, the dilator having a dilator connector electrically coupled to the one or more dilator electrodes;
wherein the second connector includes a retaining track having at least one electrical contact, the second connector being configured to mechanically and electrically couple with the dilator connector.

2. The system of claim 1, wherein the sheath lumen is configured to receive the dilator.

3. The system of claim 2, wherein the dilator includes a dilator hub, and the dilator connector is integral with the dilator hub.

4. The system of claim 1, wherein the retaining track is resilient and includes an oval shape body having a width greater than a height.

5. The system of claim 4, wherein the resilient retaining track includes a first line of symmetry and a second line of symmetry, and the at least one electrical contact is intersected by the first line of symmetry.

6. The system of claim 5, wherein the at least one electrical contract includes a conductive coating.

7. The system of claim 1, wherein the second connector includes a hemostatic valve, a cap, and a cover.

8. The system of claim 7, wherein the cap includes a retaining track guide.

9. The system of claim 1, wherein electrical connection is made between the one or more dilator electrodes and the control system when the dilator connector is received by the second connector.

10. The system of claim 1, wherein the dilator distal portion is tapered.

11. The system of claim 1, wherein the dilator connector includes a cylindrical body having an outer surface, and one or more electrical contacts positioned on the outer surface.

12. The system of claim 1, wherein the dilator connector is configured to self-align with the second connector, and wherein the dilator connector is configured to mechanically couple with the second connector in order to maintain the dilator in a desired rotational orientation relative to the sheath.

13. The system of claim 1, wherein the dilator connector includes a distal protrusion.

14. The system of claim 1, further comprising a display for displaying one or more anatomical images, parameters, and positioning information.

15. The system of claim 1, wherein the one or more sheath electrodes includes four sheath electrodes and the one or more dilator electrodes includes two electrodes.

16. A medical system, comprising:
a sheath including an elongate body having a proximal portion, a distal portion, and a lumen therebetween;
one or more sheath electrodes located on the distal portion;
a handle attached to the proximal portion, the handle having a first connector capable of electrically coupling to a control system and a second connector coaxial with the lumen; and
a dilator including an elongate body having a proximal portion and a distal portion having one or more dilator electrodes, the dilator having a dilator connector electrically coupled to the one or more dilator electrodes;
wherein the second connector includes a resilient retaining track being configured to mechanically and electrically couple with the dilator connector, and
wherein electrical connection is made between the one or more dilator electrodes and the control system when the dilator connector is received by the second connector.

17. The system of claim 16, wherein the resilient retaining track includes an oval shape body having a width greater than a height.

18. The system of claim 17, wherein the resilient retaining track includes at least one electrical contact.

19. The system of claim 18, wherein the at least one electrical contact includes a conductive coating.

20. A medical system, comprising:
a sheath including an elongate body having a proximal portion, a distal portion, and a lumen therebetween;
one or more sheath electrodes located on the distal portion;
a handle attached to the proximal portion, the handle having a first connector capable of electrically coupling to a control system and a second connector coaxial with the lumen;
a dilator including an elongate body having a proximal portion and a distal portion;
one or more dilator electrodes located on the distal portion; and
a dilator connector electrically coupled to the one or more dilator electrodes; and
a display for displaying one or more anatomical images, parameters, and positioning information;
wherein the second connector includes a resilient retaining track having at least one conductive snap, the second connector being configured to receive the dilator elongate body and to electrically couple with the dilator connector.
